SEQUENCE LISTING (6 sequences)

SEQ ID NO: 1 - DNA, 22 bp, artificial
  oligonucleotide primer
tgaaggctcatatctgtctccg

SEQ ID NO: 2 - DNA, 22 bp, artificial
  oligonucleotide primer
catcgaggacatccaagacaac

SEQ ID NO: 3 - DNA, 26 bp, Artificial
  oligonucleotide primer
gaggctctcttccagccttccttcct

SEQ ID NO: 4 - DNA, 26 bp, artificial
  oligonucleotide primer
cctgcttgctgatccacatctgctgg

SEQ ID NO: 5 - DNA, 30 bp, artificial
  3′ biotinylated oligonucleotide probe
ctgacacaggctaaaggtcatctgaagaag

SEQ ID NO: 6 - DNA, 30 bp, artificial
  antisense counterpart to SEQ ID NO5
cttcttcagatgacctttagcctgtgtcag

(19) United States
(12) Patent Application Publication
(10) Pub. No.: US 2007/0249561 A1
(43) Pub. Date: Oct. 25, 2007

(54) Pharmacological method for treatment of neuropathic pain

(76) Inventor: Bradley K. Taylor, New Orleans, LA (US)

Correspondence Address: ADAMS AND REESE LLP, 4400 ONE HOUSTON CENTER, 1221 MCKINNEY, HOUSTON, TX, 77010, US

(21) Appl. No.: 11/739,811

(22) Filed: Apr. 25, 2007

Related U.S. Application Data
(60) Provisional application No. 60/795,078, filed on Apr. 25, 2006.

(51) Int. Cl. A61K 31/675; A61K 31/4439; A61K 31/433; A61K 31/426; A61K 31/421; A61K 31/557; A61K 31/275

(52) U.S. Cl. 514/79; 514/342; 514/369; 514/352; 514/573; 514/362; 514/374; 514/521

(57) ABSTRACT

Disclosed are methods and compositions useful for treatment of neuropathic pain. In particular, the present invention provides methods of activating gamma-subtype peroxisome proliferator-activated receptors (PPARγ) to inhibit, relieve, or treat neuropathic pain.

### CROSS-REFERENCE TO RELATED APPLICATIONS

This Non-Provisional Patent Application, filed under 35 U.S.C. § 111 (a), claims the benefit under 35 U.S.C. § 119(e)(1) of U.S. Provisional Patent Application No. 60/795,078, filed under 35 U.S.C. § 111 (b) on Apr. 25, 2006, and which is hereby incorporated by reference in its entirety.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

The Sequence Listing, which is a part of the present disclosure and is submitted in conformity with 37 CFR §§ 1.821-1.825, includes a computer readable form and a written sequence listing comprising nucleotide and/or amino acid sequences of the present invention. The sequence listing information recorded in computer readable form (created 19 Apr. 2007; filename: Neuropathic_Pain_ST25; size: 4 KB) is identical to the written sequence listing below. The subject matter of the Sequence Listing is incorporated herein by reference in its entirety.

### BACKGROUND OF THE INVENTION

1. Field of the Invention

The present invention relates to methods of activating gamma-subtype peroxisome proliferator-activated receptors (PPARγ) to inhibit neuropathic pain. The invention also relates to thiazolidinediones, and to methods for treating neuropathic pain employing thiazolidinediones.

2. Description of Related Art

Pain is defined as an unpleasant bodily sensation in response to one or more sensory stimuli. Pain can be physiological or psychological in origin, and it can be either acute or chronic. Acute pain is considered an important part of the body's defense system, alerting it to injury or other conditions which can endanger health, while chronic pain appears to serve no useful purpose and only makes patients miserable.

Clinically, pain is classified further as “inflammatory” or “nociceptive” if it appears that severity of the pain is correlated with the degree of nociceptor stimulation by processes causing tissue injury (e.g., a burn or laceration). Nociceptors are specialized sensory neurons with cell bodies in dorsal root ganglia (or trigeminal ganglion), a first axonal process that terminates in peripheral tissue (e.g., the hand), and a second axonal process that terminates in the spinal cord or brainstem. They are activated by noxious insult to peripheral tissues. They have undifferentiated or “free” nerve endings, and their activation appears to involve ion channels (e.g., vanilloid receptor 1, or the “capsaicin receptor,” and other transient receptor potential channels, or TRPCs) that are activated by various stimuli including heat, cold, and chemical compounds. Activation of nociceptors, however, does not necessarily cause the perception of pain. Rather, pain perception is a product of the brain's cumulation, abstraction, and interpretation of sensory input, and nociceptors provide input to the brain via afferent fibers that terminate on neurons (including projection neurons and interneurons) in the spinal cord dorsal horn.

“Neuropathic” pain is similarly accompanied by tissue injury, but is due to direct injury to nerve fibers in the peripheral or central nervous systems. It is subcategorized as peripheral or central, depending on the location or source of the lesion initiating the neuropathic pain (e.g., in peripheral tissues or within the spinal cord, respectively). Neuropathic pain often involves mis-directed or improper neural signaling to pain centers of the central nervous system, and comprises: reflex sympathetic dystrophy syndrome, also known as complex regional pain syndrome; postherpetic neuralgia, or pain that occurs in some patients after an episode of herpes zoster (shingles); anesthesia dolorosa, or “pain in the absence of sensation,” which occurs when sensory nerves, and especially the trigeminal nerve, are damaged (surgically or traumatically) in such a way that sensation is reduced or eliminated, yet pain sensation remains; trigeminal neuralgia, or tic douloureux; human immunodeficiency virus-related neuropathic pain; post-stroke neuropathic pain; and low back pain of peripheral nerve origin (Bennett, 1998; Taylor, 2004). Neuropathic pain may also be related to or caused by: multiple sclerosis; cancer; anti-cancer drugs; nerve/plexus metastatic invasion; nerve compression; surgical injury; nerve inflammation or insult secondary to ischemia; and hereditary factors (Bennett, G. J. Hospital Practice, Vol. 33, no. 10 (Oct. 15, 1998), pp. 95-8, 101-4, 107-10 passim; Taylor, B. K. “The Pathophysiology of Neuropathic Pain” in: Neurosurgical Pain Management (Kenneth A. Follett ed., Elsevier Saunders 2004), pp. 29-37.).

Another remarkable example of neuropathic pain is “phantom limb syndrome,” which is the sensation that an amputated limb (removed surgically or traumatically) remains attached to the body and moves appropriately with one's remaining body parts (e.g., feeling the phantom limb try to shake hands when greeting someone). About 50 to 80% of amputees report phantom sensations in their amputated limbs. A majority of amputees report that the phantom sensation is painful, but also report sensations of warmth, cold, itching, burning, and compression.

Although many mechanisms of pain transmission are understood, neuropathic pain remains an important medical problem that is particularly difficult to treat. It was initially thought that neuropathic pain after amputation derived from inflammation of severed nerve endings. Attempts to alleviate such pain by performing a second amputation, shortening the stump to remove the inflamed nerve endings, often increased patients' discomfort instead, and left many with the original phantom limb sensation plus sensation from a “phantom stump.”

Pain, in general, is treated in a number of ways, including pharmacologically, psychologically, and by alternative medicine. While pharmacological approaches to management of nociceptive pain have been relatively successful, these approaches also present disadvantages such as toxicity (e.g., aspirin, ibuprofen, acetaminophen) and addiction (e.g., opiates), thus limiting their use. Neuropathic pain, however, has been largely refractory to traditional pharmacological pain management protocols, in part because the molecular mechanisms underlying the genesis and transmission of neuropathic pain are poorly understood. For example, first-line medical therapies such as gabapentin and opioids only reduce neuropathic pain by 26 to 38 percent (Gilron I. et al., New England journal of Medicine. 2005; 352(13):1324-34).

Peroxisome proliferator activated receptors (PPARs) belong to the nuclear hormone receptor superfamily of ligand-activated transcription factors, and are related to retinoid, steroid, and thyroid hormone receptors. There are three known PPAR subtypes, designated α, β/δ, and γ. When bound by their cognate ligands, PPARs form a heterodimer with retinoid receptor X (RXR), and the heterodimer complex subsequently binds specific response elements in the promoter regions of target genes. Thus, activation of PPARs leads to gene transcription and protein expression.

Agonists are compounds that bind to a receptor (e.g., PPARγ) and trigger a measurable response (e.g., phosphorylation, cellular differentiation and proliferation), mimicking the activity of an endogenous ligand (e.g., a hormone or neurotransmitter) that recognizes and binds to the same receptor. Antagonists are also compounds that bind to a receptor, but they inhibit the function of agonists. Generally, there are three types of receptor antagonists. Competitive antagonists bind reversibly to receptors, and compete with agonists (and other antagonists) for the same binding site on the receptor. Reversible non-competitive antagonists do not compete for the same binding site as agonists, yet they still function to inhibit agonist-mediated effects. Finally, irreversible antagonists bind covalently to a receptor, at the receptor binding site, and inhibit agonist-mediated effects. They are also non-competitive because they cannot be displaced by higher concentrations of agonist.

Determining whether a compound is an agonist for a particular receptor is a relatively straightforward affair, with the materials and methods required being well-known to one of ordinary skill in the art. For PPARγ, commercially-available kits (e.g., TF ELISA PPARγ Assay Kit, BioCat GmbH, Heidleberg, Germany, or LightShift Chemiluminescent EMSA Kit, Pierce Biosciences, Rockford, Ill.) facilitate the evaluation of transcription factor activation from cell nuclear extracts. For example, using the methods of such a kit, addition of a known or putative PPARγ agonist compound to cells that express PPARγ would result in selective isolation and calorimetric identification of activated PPARγRXR heterodimers, thus confirming the compound functions as an agonist. Antagonists could be identified by, for example, testing them against known agonists in the same assay. Thiazolidinediones, also called “glitazones,” are a family of compounds that have received substantial attention for their usefulness as antidiabetic agents, and include such compounds as rosiglitazone, pioglitazone, englitazone, ciglitazone, and troglitazone. Thiazolidinediones are also PPARγ agonists, and their efficacy as antidiabetic agents has been attributed to their ability to stimulate adipocyte differentiation by activating PPARγ (Lehmann et al., 1995).

The technical problem underlying the present invention was therefore to overcome these prior art difficulties by furnishing analgesic agents to manage neuropathic pain, preferably without serious risk of toxicity or addiction. The solution to this technical problem is provided by the embodiments characterized in the claims.

### BRIEF SUMMARY OF THE INVENTION

The present invention relates to methods of inhibiting or relieving neuropathic pain by administering pharmaceutical compositions to activate the gamma subtype of peroxisome proliferator-activated receptors (PPARγ).

The present invention provides methods of treating neuropathic pain in a mammal (including a human) in need of such treatment comprising administering to said mammal (e.g., a human) an effective amount of a PPARγ agonist. PPARγ agonists comprise thiazolidinediones, which have received substantial attention for their usefulness in treating diabetes, and other compounds identified by their ability to activate PPARγ. We discovered that activation of PPARγ (also called “NR1C3”) produces dramatic reduction of neuropathic pain, and so provides a useful new therapeutic avenue. Consequently, PPARγ agonists are useful for the treatment of neuropathic pain. A particularly exciting candidate for PPARγ analgesia is pioglitazone, because it can cross the blood brain barrier to affect the central nervous system directly (see, e.g., Heneka M. T. et al., Brain. 2005; 128(pt 6):1442-53; and Maeshiba Y. et al., Arzneimittel-Forschung. 1997; 47(1):29-35).

The data presented herein establishes the unexpected biological benefits achievable with PPARγ agonists, including thiazolidinediones, according to the methods of the present invention. The administration of PPARγ agonist may be selected from a variety of routes known in the art. Preferably, the administration is oral administration. Also preferably, the PPARγ may be within a tablet or a capsule.

The present invention also provides methods of treating neuropathic pain in a mammal (including a human) in need of such treatment comprising administering an effective amount of a compound or compounds of Formula I Formula I

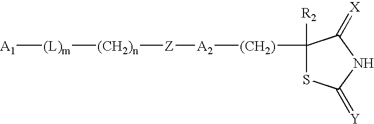

or a tautomeric form thereof and/or a pharmaceutically acceptable salt thereof, and/or a pharmaceutically acceptable solvate thereof, wherein: A₁ represents a substituted or unsubstituted aromatic heterocyclyl group; L represents O, S, or NR₁ wherein R₁ represents a hydrogen atom, an alkyl group, an acyl group, an aralkyl group wherein the aryl moiety may be substituted or unsubstituted, or a substituted or unsubstituted aryl group; m represents an integer in the range of from 0 to 1; n represents an integer in the range of from 1 to 6; Z represents O or S; A₂ represents a benzene ring having in total up to 5 substituents; R₂ represents a hydrogen atom, an alkyl, aralkyl, or aryl group; and Y and Z are, independently, O or NH.

Suitable aromatic heterocyclyl groups include substituted or unsubstituted, single or fused ring aromatic heterocyclyl groups comprising up to 4 hetero atoms in each ring selected from oxygen, sulphur, or nitrogen.

Favored aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulphur, or nitrogen.

Suitable moieties for A₁, when it represents a 5-membered aromatic heterocyclyl group, include thiazolyl and oxazoyl, especially oxazoyl.

Suitable values for A₁, when it represents a 6-membered aromatic heterocyclyl group include pyridyl or pyrimidinyl.

Suitable R₂ moieties are hydrogen and an alkyl group, including a C₁₋₆ alkyl group, for example a methyl group.

As desired, the mammal to be treated with a compound of Formula I may be a human, and the administration of PPARγ agonist may be selected from a variety of routes known in the art. Preferably, the administration is oral administration. Also preferably, the PPARγ agonist may be within a tablet or a capsule.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements.

FIG. 1A shows the dose-dependent effects over time of 15-deoxy-Δ^{12,14} prostaglandin J₂ (15-deoxy-Δ^{12,14} PGJ2, 15d-PGJ2, or simply PGJ2), an endogenous PPARγ agonist, on mechanical threshold, a behavioral sign of neuropathic pain. FIG. 1B shows the dose-dependent effects of 15-deoxy-Δ^{12,14} PGJ2 on mechanical threshold at 60 minutes after injection, and FIG. 1C shows the log dose-response curve at 60 minutes after injection.

FIG. 2A shows that the effects over time of 15-deoxy-Δ^{12,14} PGJ2 on mechanical threshold are blocked in a dose-dependent manner by co-administration of the PPARγ antagonist bisphenol A diglycidyl ether (BADGE). FIG. 2B shows the dose-dependent effects of BADGE on 15-deoxy-Δ^{12,14} PGJ2-induced elevation of mechanical threshold averaged from 30 to 90 minutes after injection. FIG. 2C shows the log dose-response curve at 60 minutes after injection.

FIG. 3A shows the dose-dependent effects over time of the PPARγ agonist rosiglitazone on mechanical threshold. FIG. 3B shows the dose-dependent effects of rosiglitazone on mechanical threshold at 60 and 90 minutes after injection. The symbols and treatments indicated in FIG. 3A correspond to the symbols of FIG. 3C, which shows the dose-dependent effects over time of rosiglitazone on cold allodynia (“allodynia” refers to pain from stimuli that normally do not invoke a pain response), and FIG. 3D shows the dose-dependent effects of rosiglitazone on cold allodynia at 60 and 90 minutes after injection.

FIG. 4A shows that the effects over time of 100 μg rosiglitazone on mechanical threshold are blocked in a dose-dependent manner by co-administration of BADGE. FIG. 4B shows the dose-dependent effects of BADGE on rosiglitazone-induced elevation of mechanical threshold at 90 minutes after injection. The symbols and treatments indicated in FIG. 4A correspond to the symbols of FIG. 4C, which shows that the effects over time of 100 μg rosiglitazone on cold allodynia are blocked in a dose-dependent manner by co-administration of BADGE. FIG. 4D shows the dose-dependent effects of BADGE on rosiglitazone-induced suppression of cold allodynia at 90 minutes after injection.

FIG. 5A shows the dose-dependent effects over time of the PPARγ agonist pioglitazone on mechanical threshold. FIG. 5B shows the dose-dependent effects over time of pioglitazone on cold allodynia.

FIG. 6 shows that rosiglitazone does not affect mechanical threshold (FIG. 6A), cold response (FIG. 6B), IR latency (FIG. 6C), or motor coordination (FIG. 6D) in animals subjected to sham SNI surgery. The symbols and treatments indicated in FIG. 6A correspond to the symbols of FIGS. 6B and C.

FIG. 7A is a melt curve for real-time PCR of PPARγ mRNA amplified from rat spleen (left-most curve), liver (middle curve), and spinal cord (right-most curve). FIG. 7B shows quantification of data from real-time PCR of PPARγ mRNA from spinal cord, liver, brain and spleen tissue.

FIG. 8 shows the results of an electrophoretic mobility shift assay (EMSA) (FIG. 8A), and an EMSA supershift assay (FIG. 8B), using a consensus PPARγ response element (SEQ ID NO:5 annealed to SEQ ID NO:6) bearing a 3′ biotin tag to probe for activated PPARγ heterodimers. FIG. 8A depicts a shift in the apparent molecular weight of the response element (the probe), representing interaction between the probe and PPARγ/RXR heterodimers. FIG. 8B shows a supershift of the apparent molecular weight of the probe, representing a complex formed by the probe, PPARγ/RXR heterodimers, and anti-PPARγ antibody.

FIG. 9 is a Western blot of nuclear extracts from rat L4-L5 lumbar spinal cord (Lanes 1-2) and rat liver (Lanes 3-4), probed with mouse anti-PPARγ monoclonal antibody (mAb) specific for the C-terminus of human PPARγ (Santa Cruz Biotechnology, Inc., Santa Cruz, Calif.). Secondary antibody was HRP-conjugated goat-anti-mouse (Santa Cruz Biotechnology, Inc.).

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

The invention features, in one aspect, a method of treating neuropathic pain in a mammal in need of such treatment, comprising administering to said mammal an effective amount of a PPARγ agonist. Preferably, the mammal in need of such treatment is a human, and the administration may be selected from the group consisting of cutaneous; endosinusial; enteral; epidural; intra-abdominal; intraarterial; intra-bladder; intrabursal; intracartilaginous; intracaudal; intracerebral; intracranial; intra-dermal; intradiscal; intradural; intraileal; intralesional; intraluminal; intramedullary; intrameningeal; intramuscular; intraocular; intra-otic; intraperitoneal; intra-portal; intraprostatic; intrapulmonary; intra-rectal; intrasinal; intra-spinal; intrathecal; intra-tumoral; intratympanic; intravascular; intravenous; intravenous bolus; intravenous drip; intravenous infusion; intraventricular; nasal inhalation; nasogastric; oral; parenteral; periarticular; peridural; perineural; pulmonary inhalation; retrobulbar; spinal; subarachnoid; subcutaneous; sublingual; systemic; topical; transdermal; ureteral; urethral; and vaginal. Preferably, the administration is oral administration. Also preferably, the PPARγ agonist may be within a tablet or a capsule.

In a preferred embodiment, the PPARγ agonist is selected from the group consisting of: 5-(3-(3-(4-phenoxy-2-propylphenoxy)propoxy)phenyl)-2,4-thiazolidinedione (TZD-18) (see, e.g., Guo Q. et al., Endocrinology. 2004; 145(4):1640-48); (2S)-2-ethoxy-3-[4-[2-(4-methylsulfonyloxyphenyl)ethoxy]phenyl]propanoic acid (tesaglitazar) (see, e.g., Hegarty B. D., Endocrinology. 2004; 145(7):3158-64); 5-[[4-[3-(5-methyl-2-phenyl-1,3-oxazol-4-yl)propanoyl]phenyl]methyl]thiazolidine-2,4-dione (darglitazone; CAS No. 141200-24-0), which has the formula

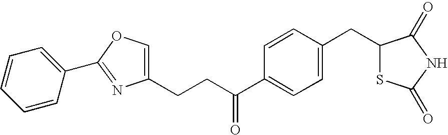

(see, e.g., Li M. et al., Bone. 2006; 39(4):796-806); 5-[[6-[(2-fluorophenyl)methoxy]naphthalen-2-yl]methyl]thiazolidine-2,4-dione (netoglitazone; GAS No. 161600-01-7), which has the formula

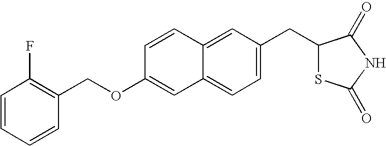

(see, e.g., Reginato M. J. et al., Journal Biological Chemistry 1998; 273(49):32679-84); 4-[(5-chloronaphthalen-2-yl)methyl]-5H-1,2,3,5-oxathiadiazole 2-oxide (WAY 120744; GAS No. 127810-07-5), which has the formula

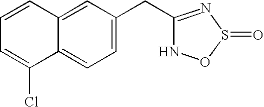

(see, e.g., Ellingboe J. W. et al., Journal of Medicinal Chemistry. 1993; 36(17):2485-93); 5-[[2-(naphthalen-2-ylmethyl)benzooxazol-5-yl]methyl]thiazolidine-2,4-dione (GAS No. 118384-10-4), which has the formula

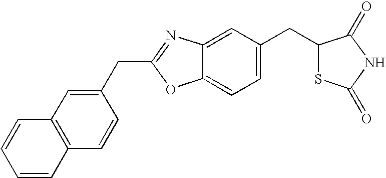

(see, e.g., Kubo H. et al., Biological & Pharmaceutical Bulletin. 1997; 20(4):460-63); and analogs thereof.

In a more preferred embodiment, the PPARγ agonist is selected from the group consisting of: 3-(2,4-dihydroxyphenyl)-5,7-dimethoxy-6-(3-methylbut-2-enyl) chromen-2-one (glycyrin); 2-cyano-3,12-dioxooleana-1,9-dien-28-oic acid (CDDO) (see, e.g., Wang Y. et al., Molecular Endocrinology. 2000; 14(10):1550-56); 5-[4-[N-(2-pyridyl)-(2S)-pyrrolidine-2-methoxyl]] phenylmethylene [thiazolidine-2,4-dione, malic acid salt] (PAT5A) (see, e.g., Vikramadithyan R. K. et al., Metabolism. 2000; 49 (11):1417-23); N-(2-benzoylphenyl)-O-[2-(methyl-2-pyridinylamino) ethyl]-L-tyrosine hydrate (GW1929) (see, e.g., Henke B. R. et al., Journal of Medicinal Chemistry. 1998; 41(25):5020-36); (S)-2-[1-carboxy-2-[4-[2-(5-methyl-2-phenyloxazol-4-yl) ethoxy]phenyl]ethylamino]benzoic acid methyl ester (GW7845) (see, e.g., Cobb J. E. et al., Journal of Medicinal Chemistry. 1998; 41 (25):5055-69); 1-O-hexadecyl-2-azelaoyl-sn-glycero-3-phosphocholine (hexadecyl azelaoyl phosphatidylcholine, azPC, azelaoyl PAF, azPAF; MDL No. MFCD03412018) (see, e.g., Davies S. S. et al., Journal of Biological Chemistry. 2001; 276:16015-23); 5-[[4-[(1-methylcyclohexyl)methoxy]phenyl]methyl]thiazolidine-2,4-dione (ciglitazone; CAS No. 74772-77-3), which has the formula

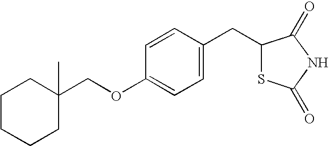

(see, e.g., Willson T. M. et al., Journal of Medicinal Chemistry. 1996; 39:665-68); 5-[[4-[(6-hydroxy-2,5,7,8-tetramethyl-chroman-2-yl)methoxy]phenyl]methyl]thiazolidine-2,4-dione (troglitazone; CAS No. 97322-87-7), which has the formula

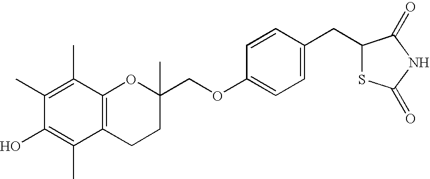

(see, e.g., Kodera Y. et al., Journal of Medicinal Chemistry. 2000; 275(43):33201-4); 5-[(2-benzylchroman-6-yl)methyl]thiazolidine-2,4-dione (englitazone; CAS No. 122228-35-7), which has the formula

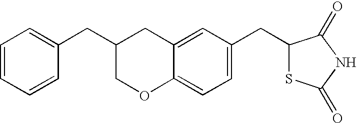

(see, e.g., Lambe K. G. et al., European Journal of Biochemistry. 1996; 239(1):1-7); 5-[[4-[2-hydroxy-2-(5-methyl-2-phenyl-1,3-oxazol-4-yl)ethoxy]phenyl]methyl]thiazolidine-2,4-dione (AD 5075; CAS No. 103788-05-2), which has the formula

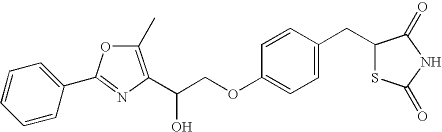

(see, e.g., Zierath J. R. et al., Endocrinology. 1998; 139(12):5034-41); (Z)-7-[(1S,5E)-5-[(E)-oct-2-enylidene]-4-oxo-1-cyclopent-2-enyl]hept-5-enoic acid (15-deoxy-Δ^{12,14}-prostaglandin J₂; CAS No. 87893-55-8), which has the formula

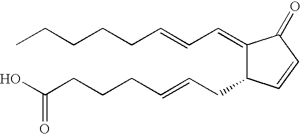

(see, e.g., Kliewer S. A. et al., Cell. 1995; 83(5):813-19); (2S)-2-[(2-benzoylphenyl)amino]-3-[4-[2-(5-methyl-2-phenyl-1,3-oxazol-4-yl) ethoxy]phenyl]propanoic acid (farglitazar; CAS No. 196808-45-4), which has the formula

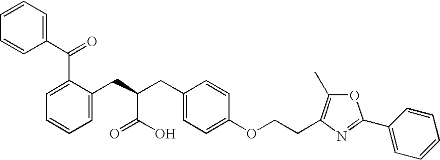

(see, e.g., Elangbam C. S. et al., Toxicologic Pathology. 2002:30(4):420-26); 1-[2-hydroxy-3-propyl-4-[4-(2H-tetrazol-5-yl)butoxy]phenyl]ethanone (tomelukast, LY171883; CAS No. 88107-10-2), which has the formula

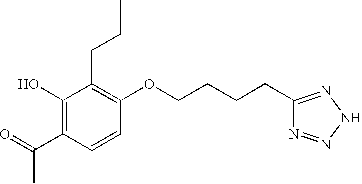

(see, e.g., Kliewer S. A. et al., Proceedings of the National Academy of Sciences of the United States of America, 1994; 91(15):7355-59); 5-[(2,4-dioxothiazolidin-5-yl)methyl]-2-methoxy-N-[[4-(trifluoromethyl)phenyl]methyl]benzamide (KRP-297; CAS No. 213252-19-8), which has the formula

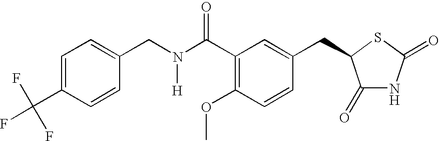

(see, e.g., Murakami K. et al., Diabetes. 1998; 47(12):1841-47); and analogs thereof.

In an even more preferred embodiment, the PPARγ is selected from the group consisting of: 5-[[4-[2-(methyl-pyridin-2-yl-amino)ethoxy]phenyl]methyl]thiazolidine-2,4-dione (rosiglitazone; GAS No. 122320-73-4), which has the formula

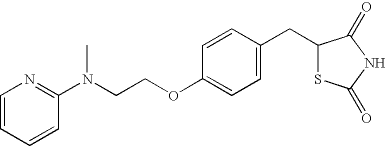

(see, e.g., Parks DJ. et al., Bioorganic Medicinal Chemist Letters. 1998; 8(24):3657-58); and 5-[[4-[2-(5-ethylpyridin-2-yl) ethoxy]phenyl]methyl]thiazolidine-2,4-dione (pioglitazone; CAS No. 111025-46-8), which has the formula

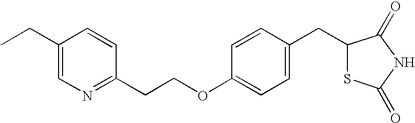

(see, e.g., Willson T. M. et al., Journal of Medicinal Chemistry 1996; 39:665-68).

In another aspect, the invention features a method of treating neuropathic pain in a mammal in need of such treatment which comprises administering to said mammal an effective amount of a PPARγ agonist of Formula I

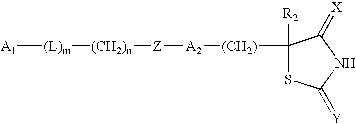

or a tautomeric form thereof and/or a pharmaceutically acceptable salt thereof, and/or a pharmaceutically acceptable solvate thereof, wherein: A₁ represents a substituted or unsubstituted aromatic heterocyclyl or heteroaryl group; L represents O, S, or NR₁ wherein R₁ represents a hydrogen atom, an alkyl group, an acyl group, an aralkyl group wherein the aryl moiety may be substituted or unsubstituted, or a substituted or unsubstituted aryl group; m represents an integer in the range of from 0 to 1; n represents an integer in the range of from 1 to 6; Z represents O or S; A₂ represents a benzene ring having in total up to 5 substituents; R₂ represents a hydrogen atom, an alkyl, aralkyl, or aryl group; and Y and Z are, independently, O or NH.

Suitable aromatic heterocyclyl groups include substituted or unsubstituted, single or fused ring aromatic heterocyclyl groups comprising up to 4 hetero atoms in each ring selected from oxygen, sulphur, or nitrogen.

Favored aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulphur, or nitrogen.

Suitable moieties for A₁, when it represents a 5-membered aromatic heterocyclyl group, include thiazolyl and oxazoyl, especially oxazoyl.

Suitable values for A₁ when it represents a 6-membered aromatic heterocyclyl group include pyridyl or pyrimidinyl.

Suitable R₂ moieties are hydrogen and an alkyl group, including a C₁₋₆ alkyl group, for example a methyl group.

Preferably, A₁ represents a moiety of Formula (a), (b), or (c)

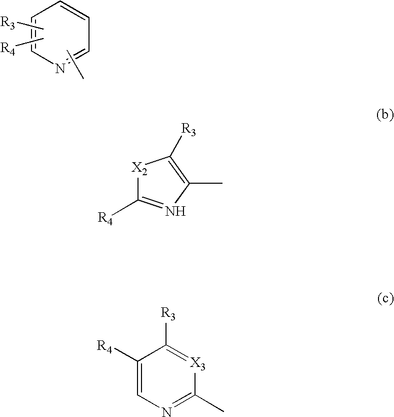

wherein: X₂ represents O or S; X₃ represents N or C; and R₃ and R₄ each independently represent a hydrogen atom, alkyl group, a substituted or unsubstituted aryl group, or (when R₃ and R₄ are each attached to adjacent carbon atoms) together with the carbon atoms to which they are attached form a benzene ring wherein each carbon atom represented by R₃ and R₄ together may be substituted or unsubstituted.

In another aspect, R₃ and R₄ represent together a moiety of Formula (d):

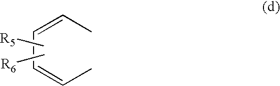

wherein R5 and R6 each independently represent hydrogen, halogen, substituted or unsubstituted alkyl, or alkoxy.

The compounds of Formula I are capable of further forming both pharmaceutically acceptable acid addition and/or base salts. All of these forms are within the scope of the present invention.

Pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glucamine (see, e.g., Berge S. M. et al., Journal of Pharmaceutical Science. 1977; 66:1-19).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner or as above. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., Berge S. M. et al., Journal of Pharmaceutical Science. 1977; 66:1-19).

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner or as above. The free acid forms differ from their respective salt forms somewhat in certain physical properties such as solubility in polar solvents, but otherwise the salts are equivalent to their respective free acid for purposes of the present invention.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereospecific synthesis techniques are employed or optically active compounds are employed as starting materials in the preparation of the compounds, individual isomers may be prepared directly; on the other hand, if a mixture of isomers is prepared, the individual isomers may be obtained by conventional resolution techniques, or the mixture may be used as it is, without resolution.

Furthermore, the thiazolidene part of the compound of Formula I can exist in the form of tautomeric isomers. All of the tautomers are represented by Formula I, and are intended to be a part of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form.

The quantity of active component in a unit dose preparation may be varied or adjusted from 0.1 mg to 100 mg preferably 0.5 mg to 100 mg according to the particular application and the potency of the active component. The composition can, if desired, also contain other compatible therapeutic agents.

The following examples are provided to demonstrate and further illustrate certain preferred embodiments and aspects of the present invention, and are not to be construed as limiting the scope thereof.

Spared nerve injury (SNI) model of neuropathic pain in rats

Male Sprague-Dawley rats (Charles Rivers Laboratories, Inc) were used, and weighed 280-320 g at the time of surgery and intrathecal catheter implantation, and 340-380 g during pharmacological testing. Animals were housed in individual cages on a 12-hour light/dark cycle starting at 6 a.m., and were given food and water ad lithium. All animal use protocols were approved by the Institutional Animal Care and Use Committee (IACUC) of Tulane University.

Neuropathic pain was modeled in rats using the spared nerve injury model (“SNI”), which is widely accepted as a model for neuropathic pain. To produce the model, rats were anesthetized with isoflurane (5% induction, then 1.5% maintenance in oxygen). As previously described, an incision was made in the skin at the level of the trifurcation of the left sciatic nerve (Decosterd et al., 2000). The overlying muscles were retracted, exposing the common peroneal, tibial, and sural nerves. The common peroneal and tibial nerves were ligated with 6-0 silk (Ethicon, Somerville, N.J.), and then the knot and adjacent nerve (2 mm) were transected. Care was taken to avoid touching the sural nerve branch. The muscle was then sutured with 4-0 absorbable sutures (Ethicon) and the wound was closed with metal clips.

At the time of nerve injury, animals were re-anesthetized with isoflurane (Baxter, Deerfield, Ill.), and then placed in a stereotaxic apparatus (Stoelting, Wood Dale, Ill.). As previously described (Malkmus et al., 2004), rats were implanted with polyethylene-10 (PE-10, Clay Adams, Sparks, Md.) intrathecal catheters. Briefly, the animal's head was flexed forward in the stereotaxic apparatus, an incision was made in the skin at the back of the head and neck, and the cisternal membrane was exposed by sharp dissection. The membrane was gently punctured with the tip of a #15 scalpel blade, and the distal end of a 7.5 cm long PE-10 catheter was passed through the opening in the cisternal membrane, into the intrathecal space. The catheter was loosely sutured to subcutaneous tissue, leaving the proximal end external to the animal and accessible to the experimenter, and the skin was then approximated using 4-0 absorbable sutures (Ethicon).

Following surgery, rats were allowed 7 days to recover prior to drug administration and experimentation. To minimize any effects of animal handling on experimental data, drugs and vehicle (saline, or saline and DMSO) were administered via remote injection. PE-10 tubing, filled with vehicle or drug, was used to connect a Hamilton microsyringe to a 30-gauge microinjector, through which 15-20 μL of vehicle or drug was delivered to the lumbar region of the spinal cord via the intrathecal catheter. Progress of the injection was visually confirmed by observing the movement of a small air bubble within the PE-10 tubing. Injectors were left in place an additional minute after fluid delivery, to minimize backflow within the catheter, and animals were then returned to the testing box.

Spared nerve injury (SNI) model of neuropathic pain in mice

Male CD1 mice (Charles Rivers Laboratories, Inc) were used, and weighed 18-22 g at the time of surgery, and 29-32 g during pharmacological testing. To produce the SNI model in mice, surgical procedures essentially identical to those used in rat were used. Unlike the rat SNI model, where an indwelling catheter was inserted surgically, vehicle or drugs were injected directly into the intrathecal space of the unanesthetized mouse using the classical method of Hylden and Wilcox (“Intrathecal morphine in mice: a new technique” Eur. J. Pharmacol. 17:313-6, 1980) to administer drugs to the intrathecal space dorsal to but not within the spinal cord. Briefly, a piece of cloth was used to restrain the mouse by cradling the iliac crest between one's thumb and forefinger. The L5-L6 spinal bones were located, and a 0.5 inch 30G hypodermic needle was inserted perpendicularly into the L4-L5 interspinous space. Upon insertion, the needle was angled about 30°-45° from the coronal (frontal) plane, and then advanced slightly. The needle angle was then adjusted gently, until paw or tail movement was evoked, whereupon the needle was held in position and 5 μL of saline or drug was administered via a 25 μL Hamilton syringe.

Pharmaceutical preparations and statistical analysis

All drug solutions were freshly prepared daily. After evaporating methyl acetate solvent under a gentle nitrogen stream, 15-deoxy-Δ^{12,14} PGJ2 was reconstituted in isotonic saline (comprising 0.9% sodium chloride in water, also called “normal saline”). Rosiglitazone was diluted to the concentrations indicated in the FIGS. with a solution of 30% dimethylsulfoxide (DMSO) and 70% isotonic saline. Bisphenol A diglycidyl ether (BADGE) was diluted to the concentrations indicated in the FIGS. with 100% DMSO. 15-deoxy-Δ^{12,14} PGJ2, rosiglitazone, BADGE, and DMSO were all obtained from Cayman Chemicals (Ann Arbor, Mich.). As used herein, the term “vehicle” refers to the solvent used to dilute the drugs, and may comprise without limitation, DMSO, saline, and water.

Data are presented as mean±standard error of the mean (SEM). Differences between means were analyzed by two-way repeated-measures analysis of variance (ANOVA), with drug treatment as the between-subjects variable and time as the repeated measure. If statistically significant differences (p<0.05) were detected, the analyses were followed by post hoc t-tests.

Behavioral tests of mechanical and cold allodynia

The procedures used to evaluate behavioral signs of mechanical and cold allodynia were essentially the same for rats as they were for mice. Animals were acclimated to a stainless steel grid within individual Plexiglas boxes for 30-60 minutes, and then tested for mechanical allodynia and cold allodynia. In all animals, mechanical allodynia was assessed using von Frey (VF) filaments (Stoelting, Inc), and was done before assessing cold allodynia.

To assess mechanical allodynia, the plantar region of each hind paw was stimulated with an incremental series of 8 different VF hairs (monofilaments of logarithmically varying stiffness). In SNI rats and mice, the stimulus region was localized to the sural innervation territory of the lateral aspect of the plantar hind paw. The 50% withdrawal threshold (“mechanical” threshold) was determined using the up-down method of Dixon, modified by Chaplan et al. (Chaplan et al., 1994). First, an intermediate von Frey monofilament (e.g., for rats, number 4.31 was used, which exerts a force of 2.0 g) was applied perpendicular to the plantar skin, causing a slight bending of the hair. In the event of a positive response (defined as rapid withdrawal of the paw within 6 seconds), a smaller filament was tested. In the event of a negative response, a larger filament was tested. The mechanical threshold is the smallest amount of force required to evoke a positive response. Less than 5% animals did not develop mechanical allodynia on the day of pharmacological testing after nerve injury. In such cases von Frey testing was either terminated, or that data was not included in the final analysis.

Cold allodynia was evaluated by applying a drop of acetone to the plantar paw of control and SNI rats. Acetone was applied via a syringe connected to PE-90 tubing, flared at the tip to a diameter of 3.5 mm. Surface tension maintained the volume of the drop to 10-12 μL. The length of time the animal lifted or shook its paw was recorded. The duration of paw withdrawal was recorded for 30 seconds, and three observations were averaged. Less than 5% of SNI animals did not develop cold allodynia on the day of pharmacological testing after nerve injury. In such cases acetone testing was either terminated, or that data was not included in the final analysis.

Rotarod testing

To evaluate the effect of PPARγ agonists on proprioceptive function, rats were placed on an accelerating rotarod (Stoelting, Wood Dale, III). The rotating rod was subdivided into four compartments for the simultaneous assessment of four animals. Initially rotating at a rate of 4 rpm, the system was adjusted gradually over 10 minutes to a maximum speed of 40 rpm. The time that a rat remained on the rotating rod without falling off was recorded—the rotarod duration. After sham SNI surgery and one day before testing, animals were acclimated to the rotarod for 5-10 trials, yielding average latencies of approximately 3 minutes. On the day of testing, rotarod duration was measured three times prior to drug or vehicle administration, and the times averaged. After drug administration, rotarod duration was measured in triplicate at 60 and 120 minutes from the time of administration, with durations averaged for each time point.

### EXAMPLE 1

Suppression of mechanical allodynia by 15-deoxy-Δ^{12,14} PGJ2

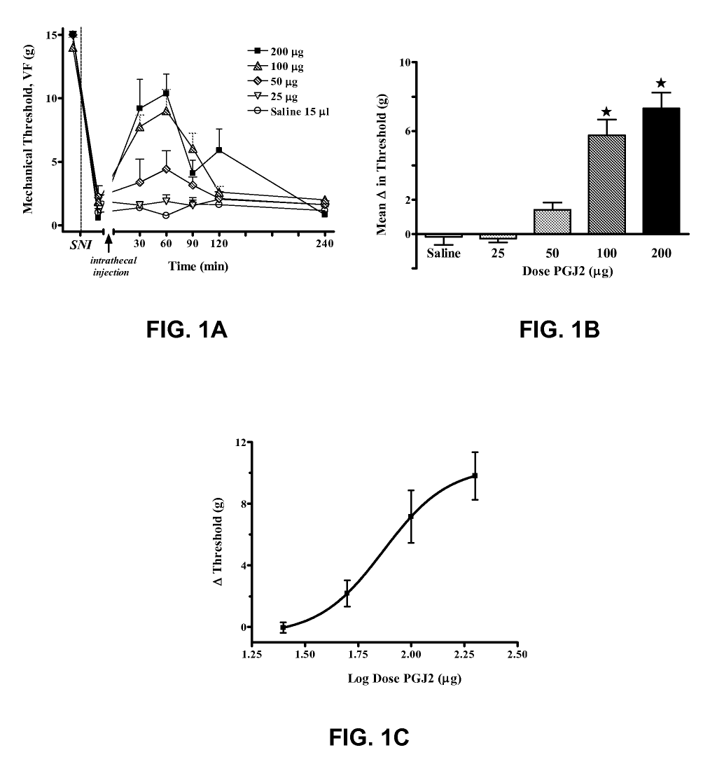

As seen in FIG. 1A, neuropathic pain was modeled in rats, and mechanical threshold to hindpaw withdrawal was tested, as described above. In the control group, injected intrathecally with 10 μL saline alone, SNI rats showed dramatically reduced latency to hindpaw withdrawal as compared with baseline measurements before SNI surgery. 15-deoxy-Δ^{12,14} PGJ2 administered intrathecally reduced behavioral signs of neuropathic pain (i.e., increased mechanical threshold, reduced the tactile allodynia component of neuropathic pain) in a dose-dependent and reversible manner, increasing latency to hindpaw withdrawal, F(4,154)=18.8, p<0.001. The analgesic effect began within 30 minutes after injection, peaked at about 60 minutes, and dissipated after four hours, suggesting that the analgesic effects of 15-deoxy-Δ^{12,14} PGJ2 do not occur via a neurotoxic mechanism. FIG. 1B summarizes the data of FIG. 1A as an average of the 30 to 90 minute data, showing the dose-dependent effect of 15-deoxy-Δ^{12,14} PGJ2. FIG. 1C is a log dose-response curve for 15-deoxy-Δ^{12,14} PGJ2, showing an ED₅₀ of 73.97 μg. “ED₅₀” is the dose of a drug that is pharmacologically effective, according to the chosen measurement (here, hindpaw withdrawal), for 50% of the population that was administered the drug. Five to seven rats were used in each group, and stars denote p<0.05 versus vehicle controls. Data presented are mean±SEM.

### EXAMPLE 2

15-deoxy-Δ^{12,14} PGJ2 suppression of neuropathic pain is mediated by PPARγ

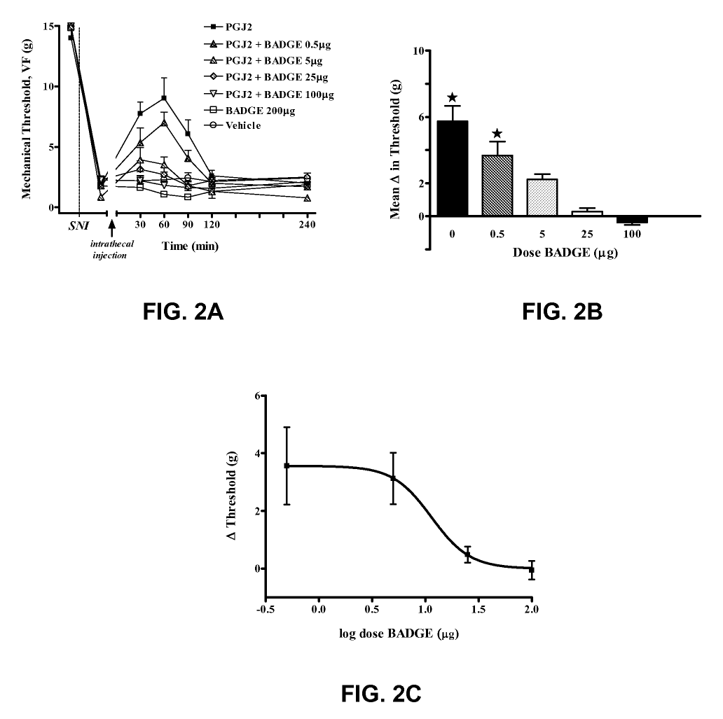

As seen in FIG. 2A, neuropathic pain was modeled in rats, and mechanical threshold to hindpaw withdrawal was tested, as described above. In the control group, treated with dimethylsulfoxide (DMSO) alone, SNI rats showed dramatically reduced latency to hindpaw withdrawal as compared with baseline measurements before SNI surgery. As in EXAMPLE 1, administration of 100 μg 15-deoxy-Δ^{12,14} PGJ2 alone increased latency to hindpaw withdrawal. Intrathecal co-administration of the PPARγ antagonist bisphenol A diglycidyl ether (BADGE) eliminated the analgesic effects of 15-deoxy-Δ^{12,14} PGJ2, F(6,33)=16.1, p<0.001, while BADGE administered alone did not have a significant effect on hindpaw withdrawal latency. FIG. 2B summarizes the data of FIG. 2A, plotted as the average of the 30-90 minute timepoints, showing the dose-dependent effect of BADGE on 15-deoxy-Δ^{12,14} PGJ2 suppression of neuropathic pain. FIG. 2C is a log dose-response curve for BADGE, showing an ED₅₀ of 11.44 μg. Four to eight rats were used in each group, and stars denote p<0.05 versus vehicle controls. Data presented are mean±SEM.

### EXAMPLE 3

Suppression of mechanical and cold allodynia by rosiglitazone

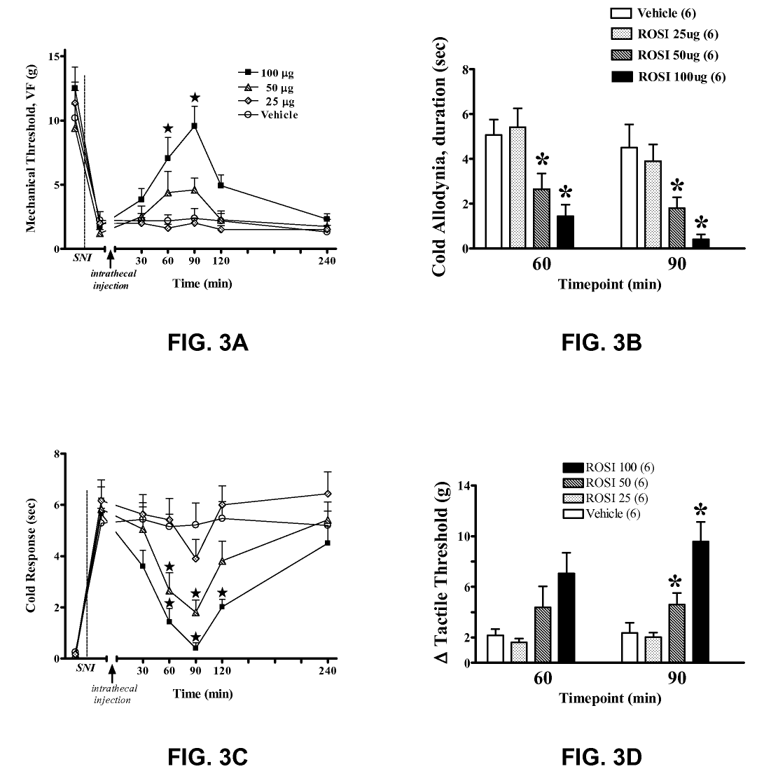

As seen in FIG. 3A, neuropathic pain was modeled in rats, and mechanical threshold to hindpaw withdrawal was tested, as described above. In the control group, treated intrathecally with vehicle (DMSO plus saline) alone, SNI rats showed dramatically reduced latency to hindpaw withdrawal (e.g., mechanical, or tactile allodynia, a key correlate of chronic neuropathic pain) as compared with baseline measurements before SNI surgery. Rosiglitazone administered intrathecally reduced behavioral signs of neuropathic pain in a dose-dependent and reversible manner, increasing latency to hindpaw withdrawal, F(3,140)=12.5, p<0.0001. The analgesic effect began within 30 minutes after injection, peaked at about 90 minutes, and dissipated after four hours, militating against a neurotoxic mechanism of action. FIG. 3B summarizes the data of FIG. 3A, at 60 and 90 minutes after the treatment indicated, showing the dose-dependent effects of rosiglitazone. Numbers in parentheses indicate the number of rats in each group, and asterisks denote p<0.05 versus vehicle controls. Data presented are mean±standard error of the mean (SEM.)

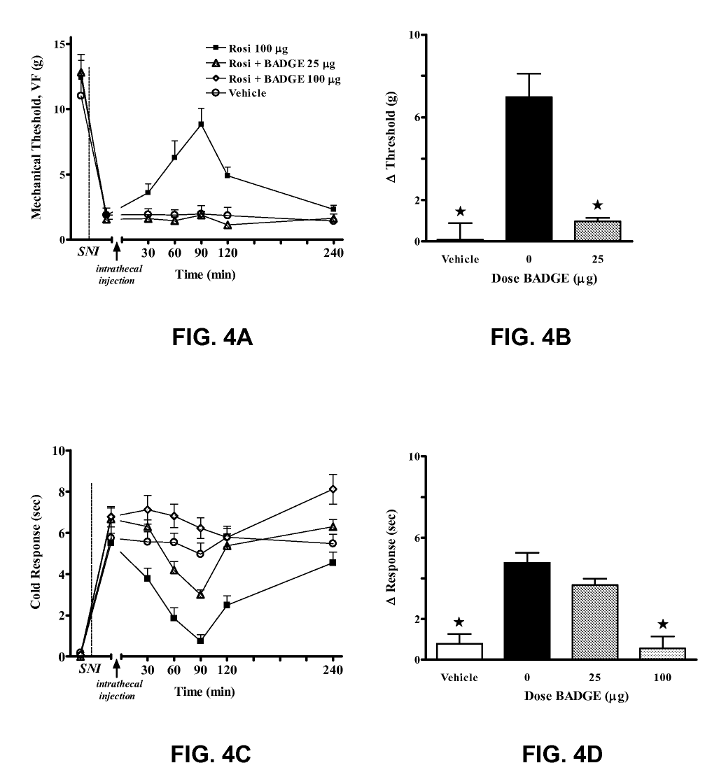

As shown in FIG. 3C, neuropathic pain was modeled in rats, and hindpaw withdrawal duration was tested, as described above. In this test a drop of acetone was applied to the hindpaw. As the acetone evaporates, a cool temperature results. An extended paw withdrawal response after application of acetone is considered a sign of cold allodynia. Comparison of pre- and post-surgery data points (pre-SNI and time 0, respectively) reveals that the duration of the paw withdrawal response after application of acetone was dramatically increased after SNI. Intrathecal rosiglitazone decreased cold hypersensitivity in a dose-dependent and reversible manner, F(3,140)=19.2, p<0.0001. The analgesic effects began within 30 minutes after injection, were most pronounced at approximately 90 minutes, and dissipated after four hours. FIG. 3D summarizes the data of FIG. 4A, at 60 and 90 minutes, showing the dose-dependent effects of rosiglitazone. Numbers in parentheses indicate the number of rats in each group, and asterisks denote p<0.05 versus vehicle controls. Data presented are mean±standard error of the mean (SEM).

### EXAMPLE 4

Rosiglitazone suppression of mechanical and cold allodynia is mediated by PPARγ

Neuropathic pain was modeled in rats, and both mechanical threshold (FIGS. 4A, B) and cold response (FIGS. 4C, D) to hindpaw withdrawal were tested as described above. In the control group of FIG. 4A, treated intrathecally with vehicle (DMSO plus saline) alone, SNI rats showed dramatically reduced latency to hindpaw withdrawal (e.g., mechanical, or tactile allodynia, a key correlate of chronic neuropathic pain) as compared with baseline measurements before SNI surgery. As in EXAMPLE 3, Rosiglitazone (100 μg) administered intrathecally reduced behavioral signs of neuropathic pain in a reversible manner, increasing latency to hindpaw withdrawal (FIG. 4A). Intrathecal co-administration of the PPARγ antagonist bisphenol A diglycidyl ether (BADGE) eliminated the analgesic effects of 100 μg rosiglitazone on mechanical hypersensitivity in a dose-dependent manner, F(2,119)=22, p<0.0001. FIG. 4B summarizes the data of FIG. 4A, showing the dose-dependent effect of BADGE on rosiglitazone-mediated suppression of mechanical allodynia. FIG. 4C shows that intrathecal rosiglitazone (100 μg) decreased cold hypersensitivity in a reversible manner, and intrathecal co-administration of BADGE dose-dependently eliminated the analgesic effects of rosiglitazone, F(2,119)=46, p<0.0001. FIG. 4D summarizes the data of FIG. 4C, showing the dose-dependent effect of BADGE on rosiglitazone-mediated suppression of cold allodynia. Stars denote p<0.05 versus vehicle controls. Data presented are mean±SEM.

### EXAMPLE 5

Suppression of mechanical and cold allodynia by pioglitazone

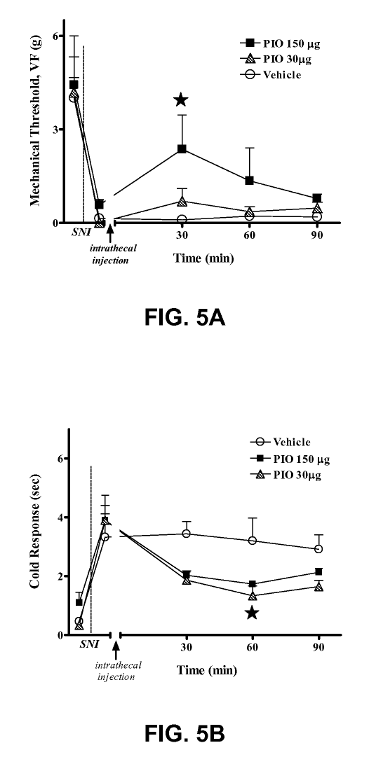

As seen in FIG. 5A, neuropathic pain was modeled in mice, and mechanical threshold to hindpaw withdrawal was tested, as described above. In the control group, treated intrathecally with vehicle (saline) alone, SNI mice showed dramatically reduced threshold to hindpaw withdrawal evoked by von Frey hairs (e.g., mechanical, or tactile allodynia, a key correlate of chronic neuropathic pain) as compared with baseline measurements before SNI surgery. Pioglitazone administered intrathecally reduced behavioral signs of neuropathic pain in a dose-dependent and reversible manner, increasing threshold to hindpaw withdrawal, F(2,60)=4.3, p<0.05. The anti-allodynic effect of the 150 μg dose began within and peaked at 30 minutes after injection, and dissipated after 90 min, arguing against a neurotoxic mechanism of action. Three to eight mice were used for each testing group, and asterisks denote statistically significant difference (p<0.05) versus vehicle controls. Data presented are mean±standard error of the mean (SEM).

As shown in FIG. 5B, neuropathic pain was modeled in mice, and hindpaw withdrawal duration was tested, as described above. In this test, a drop of acetone was applied to the hindpaw. As the acetone evaporates, a cool temperature results. A persistent foot withdrawal response after application of acetone is considered a sign of cold allodynia. Comparison of pre- and post-surgery data points (pre-SNI and time 0, respectively) reveals that the duration of the paw withdrawal response after application of acetone was dramatically increased after SNI. Intrathecal pioglitazone decreased cold hypersensitivity in a dose-dependent and reversible manner, F(2,60)=4.6, p<0.05. The analgesic effects began within 30 minutes after injection, were most pronounced at approximately 60 minutes, and began to dissipate within 90 minutes. Three to seven mice were used for each testing group, and asterisks denote statistically significant differences (p<0.05) versus vehicle controls. Data presented are mean±standard error of the mean (SEM).

### EXAMPLE 6

Rosiglitazone does not alter motor coordination, von Frey threshold, or cold withdrawal response in control rats

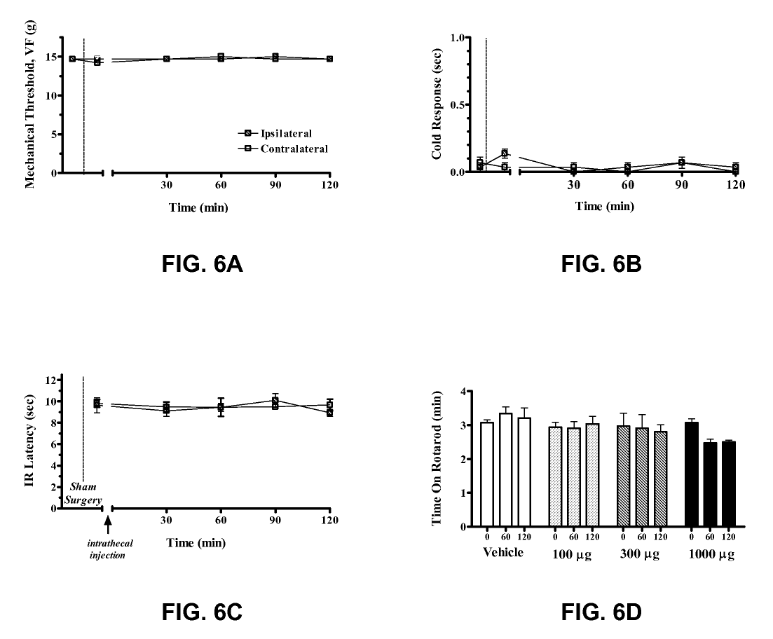

In EXAMPLES 1-4 above, neither 15-deoxy-Δ^{12,14} PGJ2 nor rosiglitazone produced gross behavioral changes or other obvious deleterious effects. To assess more subtle behavioral effects, we evaluated the effects of rosiglitazone on motor coordination and sensory thresholds in uninjured rats. As illustrated in FIG. 6, rosiglitazone does not affect mechanical threshold (FIG. 6A), cold response (FIG. 6B), latency to paw withdrawal response to an infrared heat IR) stimulus (FIG. 6C), or motor coordination (FIG. 6D) in animals subjected to sham SNI surgery (p>0.05). Only a supra-maximal dose of 1000 μg reduced motor coordination, but this effect was small, not statistically significant, and not associated with gross behavioral changes or other obvious deleterious effects.

### EXAMPLE 7

PPARγ mRNA is present in spinal cord

Rats were terminally anesthetized with ketamine and xylazine, and liver, brain, spleen and lumbar spinal cord tissue was removed. Samples were placed in RNAlater tissue storage reagent (Ambion, Austin, Tex.), and stored at 4° C. overnight. Isolation of total RNA was performed using the RiboPure Kit (Ambion, Austin, Tex.) according to the manufacturer's instructions. Purity and concentration of resulting samples was determined spectrophotometrically. Next, cDNA was prepared from 2 μg of total RNA by reverse transcription using the iScript cDNA Synthesis Kit (Bio-Rad, Hercules, Calif.) according to the manufacturer's instructions. cDNA samples were diluted 1:10 in DNase- and RNase-free water prior to further analysis.

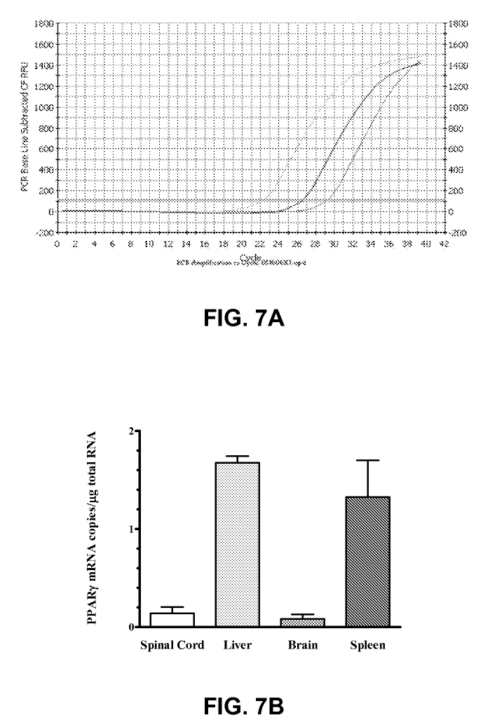

Quantitative real-time PCR was performed using the iCycler iQ Real Time Detection System (Bio-Rad, Hercules, Calif.). Gene specific primer sequences are as follows: PPARγ, forward primer 5′-TGAAGGCTCATATCTGTCTCCG-3′ (SEQ ID NO:1); PPARγ reverse primer 5′-CATCGAGGACATCCAAGACAAC-3′ (SEQ ID NO:2); β-Actin forward primer 5′-GAGGCTCTCTTCCAGCCTTCCTTCCT-3′ (SEQ ID NO:3); and β-Actin reverse primer 5′-CCTGCTTGCTGATCCACATCTGCTGG-3′. PCR reactions were carried out using 5 μL of cDNA, 10 μM of each primer, and 2× SYBR Green Supermix (Bio-Rad, Hercules, Calif.) in 25 μL reactions. Thermal cycling conditions were 95° C. for 3 min, followed by 40 cycles of 95° C. for 20 sec and 61.5° C. for 1 min. A final melting curve verified single product formation (FIG. 7A). Gene starting quantity was based on the cycle threshold (Ct) method. A control cDNA dilution series of known concentration was created for each gene to establish a standard curve, plotting the logarithm of the standard concentration against the Ct values. Unknown samples were quantitated from measured Ct values by interpolation, using the regression equation. Each value was normalized to Actin, a housekeeping gene, to control for the amount of input cDNA. Change between spared nerve injured (SNI) and sham animals was determined to be significant by the Student's t test, using a p-value of less than 0.05.

Expression of PPARγ mRNA in the liver, brain, spleen, and spinal cord was determined by quantitative real time PCR, demonstrating for the first time that PPARγ mRNA is present in the spinal cord. The optimized PCR conditions described above ensured the linearity of the serial dilutions and the efficient amplification of a single PCR product. PPARγ mRNA was found to be nearly 12-fold higher in liver and nearly 10-fold higher in spleen when compared to spinal cord (p<0.05). No significant differences were observed in PPARγ mRNA levels between brain and spinal cord tissue (FIG. 7B).

### EXAMPLE 8

PPARγ electrophoretic mobility shift assay (EMSA)

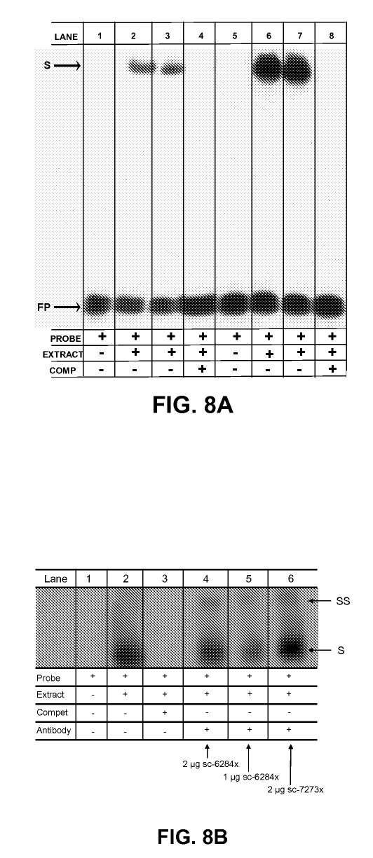

The interaction between activated PPARγ heterodimers and a consensus PPAR response element was studied (FIGS. 8A, 8B) with an electrophoretic mobility shift assay (EMSA, also called a gel shift, band shift, or gel retardation assay). Briefly, nuclear extracts from rat L4-L5 spinal cord and rat liver were obtained using a NE-PER Nuclear and Cytoplasmic Extraction Reagents kit (Pierce) according to the manufacturer's instructions, and a consensus PPAR response element 5′-CTGACACAGGCTAAAGGTCATCTGAAGAAG-3′ (SEQ ID NO:5) bearing a 3′ biotin tag was prepared. Before sample loading, 4-20% Ready Gel® TBE (Tris-buffered EDTA) gels (Bio-Rad) were electrophoresed for 1 hour at 100 V. Prior to experiments, and because PPARγ heterodimers bind double- but not single-stranded DNA, the biotinylated PPAR response element was annealed to its non-biotinylated antisense partner (SEQ ID NO:6) for 20 minutes at room temperature. Together, SEQ ID NO:5 and SEQ ID NO:6 form the “probe.” Samples were prepared using a LightShift Chemiluminescent EMSA kit (Pierce) in accord with the manufacturer's instructions. To make the samples, the following components were added to disposable microfuge tubes in the following order: ultrapure water (volume-adjusted to yield 20 μL total sample volume), 2 μL of 10× binding buffer, 1 μL polydI-dC, 1 μL 50% glycerol, 1 μL 1% NP-40, 1 μL of 1 M KCl, 1 μL 100 mM MgCl₂, 1 μL 200 mM EDTA, pH 8.0, 4 pmol unlabeled probe, 2 μL nuclear extract, 20 fmol biotin-labeled probe, and (where indicated) 1-2 μg PPARγ antibody (sc-6284× or sc-7273, Santa Cruz). Samples were then incubated at room temperature for 20 minutes, after which 5 μL of loading buffer was added. Fifteen μL of each sample was loaded into the pre-electrophoresed gel and resolved for approximately 90 minutes (or to optimal specific oligonucleotide resolution). DNA was then transferred from the gel to a Zetaprobe membrane (Bio-Rad), UV cross-linked (Fisher Scientific), probed with streptavidin-horseradish peroxidase (HRP) conjugate, and incubated with LightShift chemiluminescent substrate (Pierce, Rockford, Ill.). FIG. 8A shows results for spinal cord (lanes 1-4) and liver (lanes 5-8). Lanes 1 and 5 show the location of the unbound biotinylated probe alone (free probe, “FP”), while lanes 2-3 and 6-7 (lumbar spinal cord and liver, respectively) illustrate examples of shift bands (S), indicating that PPAR/RXR heterodimers are bound to the probe. Lanes 4 and 8 (lumbar spinal cord and liver, respectively) demonstrate specificity of the nuclear protein complex (likely a PPARγ heterodimer) to the oligonucleotide probe. In lanes 4 and 8, a competitor oligonucleotide (COMP), identical in sequence to SEQ ID NO:5 but bearing no biotin tag), was added to incubation reactions in high molar excess relative to the labeled (biotinylated) probe. Disappearance of the shift signal (S) in lanes 4 and 8 indicates that all functional transcriptional complexes are bound to unlabeled probe rather than the biotin labeled probe.

To further demonstrate the specificity of the consensus PPAR response element (probe) for heterodimer complexes involving PPARγ, a supershift assay using nuclear extracts of lumbar spinal cord was performed (FIG. 8B). When performing an EMSA with a complex mixture of proteins (e.g., nuclear extracts), one must take further steps to determine the identity of the protein bound to the probe, and the experiment described for FIG. 8A was expanded accordingly. Conditions for lanes 1-3 were the same as described above for lanes 1-3 of FIG. 8A. Lane 2 illustrates the appearance of a shift (S) band upon incubation of probe with nuclear extract, as was shown in lanes 2-3 of FIG. 8A. Lane 3 shows that the heterodimer complex binds preferably to the probe DNA sequence, as it can be competed off with excess unlabeled probe. Antibodies specific for PPARγ (sc-6284× or sc-7273, Santa Cruz) were added to sample mixtures. Lanes 4-6 of FIG. 8B show that the shift band (S) is shifted further up (SS) in the gel in samples where anti-PPARγ antibody was added. The supershift (SS) bands of lanes 4-6 illustrate that three unique anti-PPARγ antibody treatments (1 μg and 2 μg of sc-6284× and 2 μg of sc-7273×) further decrease electrophoretic mobility of the probe. This indicates that functional PPARγ is a component of the complex, derived from spinal cord nuclear extracts, bound to the probe.

### EXAMPLE 9

PPARγ protein is expressed in liver and spinal cord

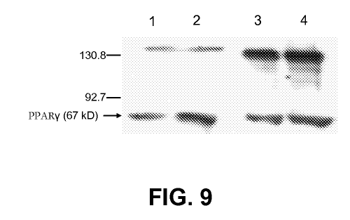

FIG. 9 is a Western blot of nuclear extracts from rat L4-L5 lumbar spinal cord (Lanes 1-2) and rat liver (Lanes 3-4), probed with mouse anti-PPARγ monoclonal antibody (mAb) specific for the C-terminus of human PPARγ (Santa Cruz Biotechnology, Inc., Santa Cruz, Calif.). Secondary antibody was HRP-conjugated goat-anti-mouse (Santa Cruz Biotechnology, Inc.). Nuclear extracts were obtained using a NE-PER Nuclear and Cytoplasmic Extraction Reagents kit (Pierce) according to the manufacturer's instructions. Extract samples were diluted with 2% Sample Buffer (Rockland, Gilbertsville, Pa.) to a final concentration of 12 μg total protein in 15 μl solution, while ensuring that the final buffer concentration was not less than 1%. Samples were then boiled for 5 minutes and subsequently loaded on a 10% Tris-HCl minigel (Bio-Rad) into a mini-electrophoresis chamber (Bio-Rad). Gels were run for approximately 90 minutes at 90 V, which provided maximum resolution around 67 kD (the apparent molecular weight of PPARγ). Proteins were transferred at 20 V for 1 hour to polyvinylidine fluoride (PVDF) membrane (Millipore, Bedford, Mass.), blocked in buffer containing 5% non-fat dry milk, probed with the antibodies described above, and visualized by chemiluminescence (SuperSignal West Pico Substrate, Pierce). FIG. 9 demonstrates that PPARγ protein is present in liver, as previously described, and—more importantly—shows for the first time that PPARγ protein is also present in spinal cord.

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A method of treating neuropathic pain in a mammal in need of such treatment which comprises administering to said mammal an effective amount of a PPARγ agonist.

2. The method of claim 1 wherein said mammal in need of such treatment is a human.

3. The method of claim 2 wherein the administration is selected from the group consisting of cutaneous, endosinusial, enteral, epidural, intra-abdominal, intraarterial, intra-bladder, intrabursal, intracartilaginous, intracaudal, intracerebral, intracranial, intra-dermal, intradiscal, intradural, intraileal, intralesional, intraluminal, intramedullary, intrameningeal, intramuscular, intraocular, intra-otic, intraperitoneal, intra-portal, intraprostatic, intrapulmonary, intra-rectal, intrasinal, intra-spinal, intrathecal, intra-tumoral, intratympanic, intravascular, intravenous, intravenous bolus, intravenous drip, intravenous infusion, intraventricular, nasal inhalation, nasogastric, oral, parenteral, periarticular, peridural, perineural, pulmonary inhalation, retrobulbar, spinal, subarachnoid, subcutaneous, sublingual, systemic, topical, transdermal, ureteral, urethral, and vaginal.

4. The method of claim 3 wherein the administration is oral administration.

5. The method of claim 4 wherein the PPARγ agonist is within a tablet or capsule.

6. The method of claim 5 wherein said PPARγ agonist is selected from:
a) 5-[[4-[2-(methyl-pyridin-2-yl-amino) ethoxy]phenyl]methyl]thiazolidine-2,4-dione;
b) 5-[[4-[2-(5-ethylpyridin-2-yl) ethoxy]phenyl]methyl]thiazolidine-2,4-dione;
c) 5-[[4-[(1-methylcyclohexyl)methoxy]phenyl]methyl]thiazolidine-2,4-dione;
d) 5-[[4-[(6-hydroxy-2,5,7,8-tetramethyl-chroman-2-yl)methoxy]phenyl]methyl]thiazolidine-2,4-dione;
e) 5-[(2-benzylchroman-6-yl)methyl]thiazolidine-2,4-dione;
f) 5-[[4-[2-hydroxy-2-(5-methyl-2-phenyl-1,3-oxazol-4-yl) ethoxy]phenyl]methyl]thiazolidine-2,4-dione;
g) (Z)-7-[(1S,5E)-5-[(E)-oct-2-enylidene]-4-oxo-1-cyclopent-2-enyl]hept-5-enoic acid;
h) (2S)-2-[(2-benzoylphenyl) amino]-3-[4-[2-(5-methyl-2-phenyl-1,3-oxazol-4-yl)ethoxy]phenyl]propanoic acid;
i) 1-O-hexadecyl-2-azelaoyl-sn-glycero-3-phosphocholine;
j) 1-[2-hydroxy-3-propyl-4-[4-(2H-tetrazol-5-yl)butoxy]phenyl]ethanone;
k) 5-[(2,4-dioxothiazolidin-5-yl)methyl]-2-methoxy-N-[[4-(trifluoromethyl)phenyl]methyl]benzamide;
l) 3-(2,4-dihydroxyphenyl)-5,7-dimethoxy-6-(3-methylbut-2-enyl) chromen-2-one;
m) 2-cyano-3,12-dioxooleana-1,9-dien-28-oic acid;
n) 5-[4-[N-(2-pyridyl)-(2S)-pyrrolidine-2-methoxyl]]phenylmethylene [thiazolidine-2,4-dione, malic acid salt];
o) N-(2-benzoylphenyl)-O-[2-(methyl-2-pyridinylamino)ethyl]-L-tyro sine hydrate;
p) (S)-2-[1-carboxy-2-[4-[2-(5-methyl-2-phenyloxazol-4-yl)ethoxy]phenyl]ethylamino]benzoic acid methyl ester;
q) 5-[[4-[3-(5-methyl-2-phenyl-1,3-oxazol-4-yl)propanoyl]phenyl]methyl]thiazolidine-2,4-dione;
r) 5-[[6-[(2-fluorophenyl)methoxy]naphthalen-2-yl]methyl]thiazolidine-2,4-dione;
s) 4-[(5-chloronaphthalen-2-yl)methyl]-5H-1,2,3,5-oxathiadiazole 2-oxide;
t) 5-[[2-(naphthalen-2-ylmethyl)benzooxazol-5-yl]methyl]thiazolidine-2,4-dione;
u) 5-(3-(3-(4-phenoxy-2-propylphenoxy)propoxy)phenyl)-2,4-thiazolidinedione; and
v) (2S)-2-ethoxy-3-[4-[2-(4-methylsulfonyloxyphenyl)ethoxy]phenyl]propanoic acid.

7. A method of treating neuropathic pain in a mammal in need of such treatment which comprises administering to said mammal an effective amount of a compound of the formula 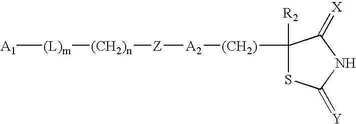
or a tautomeric form thereof and/or a pharmaceutically acceptable salt thereof, and/or a pharmaceutically acceptable solvate thereof, wherein:
a) A₁ represents a substituted or unsubstituted aromatic heterocyclyl or heteroaryl group selected from the group consisting of:
i) substituted or unsubstituted, single or fused ring aromatic heterocyclyl or heteroaryl groups comprising up to 4 hetero atoms in each ring selected from oxygen, sulphur, and nitrogen;
ii) substituted or unsubstituted single ring aromatic heterocyclyl or heteroaryl groups having 4 to 7 ring atoms, preferably 5 or 6 ring atoms;
iii) aromatic heterocyclyl or heteroaryl groups comprising 1, 2, or 3 heteroatoms, especially 1 or 2, selected from oxygen, sulphur, or nitrogen;
b) L represents O, S, or NR₁ wherein R₁ represents a hydrogen atom, an alkyl group, an acyl group, an aralkyl group, wherein the aryl moiety may be substituted or unsubstituted, or a substituted or unsubstituted aryl group;
c) m represents an integer in the range of from 0 to 1;
d) n represents an integer in the range of from 1 to 6;
e) Z represents O or S;
f) A₂ represents a benzene ring having in total up to 5 substituents;
g) R₂ represents a hydrogen atom, an alkyl, aralkyl, or aryl group;
h) Y represents O or NH; and
i) Z represents O or NH.

8. The method of claim 7 wherein said mammal in need of such treatment is a human.

9. The method of claim 8 wherein the administration is selected from the group consisting of cutaneous, endosinusial, enteral, epidural, intra-abdominal, intraarterial, intra-bladder, intrabursal, intracartilaginous, intracaudal, intracerebral, intracranial, intra-dermal, intradiscal, intradural, intraileal, intralesional, intraluminal, intramedullary, intrameningeal, intramuscular, intraocular, intra-otic, intraperitoneal, intra-portal, intraprostatic, intrapulmonary, intra-rectal, intrasinal, intra-spinal, intrathecal, intra-tumoral, intratympanic, intravascular, intravenous, intravenous bolus, intravenous drip, intravenous infusion, intraventricular, nasal inhalation, nasogastric, oral, parenteral, periarticular, peridural, perineural, pulmonary inhalation, retrobulbar, spinal, subarachnoid, subcutaneous, sublingual, systemic, topical, transdermal, ureteral, urethral, and vaginal.

10. The method of claim 9 wherein the administration is oral administration.

11. The method of claim 10 wherein the compound is within a tablet or capsule.
